(19) United States
(12) Patent Application Publication
(10) Pub. No.: US 2010/0075347 A1
(43) Pub. Date: Mar. 25, 2010

(54) METHODS OF DETECTION USING ACOUSTO-MECHANICAL DETECTION SYSTEMS

(76) Inventors: Sridhar V. Dasaratha, Bangalore (IN); Samuel J. Gason, Victoria (AU); Paul N. Holt, Hudson, WI (US)

Correspondence Address: 3M INNOVATIVE PROPERTIES COMPANY, PO BOX 33427, ST. PAUL, MN, 55133-3427, US

(21) Appl. No.: 12/375,731

(22) PCT Filed: Aug. 15, 2007
(86) PCT No.: PCT/US07/75948

§ 371 Date: Nov. 13, 2009

Related U.S. Application Data
(60) Provisional application No. 60/822,862, filed on Aug. 18, 2006.
(60) Provisional application No. 60/882,816, filed on Dec. 29, 2006.

(51) Int. Cl. G01N 33/569; G01N 33/543; G01N 33/554

(52) U.S. Cl. 435/7.33; 436/518; 435/7.32

(57) ABSTRACT

Methods for detecting target biological analytes within sample material using acousto-mechanical energy generated by a sensor are disclosed. The acousto-mechanical energy may be provided using an acousto-mechanical sensor, e.g., a surface acoustic wave sensor such as, e.g., a shear horizontal surface acoustic wave sensor (e.g., a LSH-SAW sensor). The detection of the target biological analytes in sample material are enhanced by coupling of the target biological analyte (e.g., through the use of magnetic particles), application of a magnetic field to draw the target analyte to the sensor surface, and subsequent removal of the magnetic field before measuring detection.

### GOVERNMENT RIGHTS

The U.S. Government may have certain rights to this invention under the terms of DAAD 13-03-C-0047 granted by Department of Defense.

### BACKGROUND

In the case of acousto-mechanical sensors, many biological analytes are introduced to the sensors in combination with a liquid carrier. The liquid carrier may undesirably reduce the sensitivity of the acousto-mechanical detection systems. Furthermore, the selectivity of such sensors may rely on properties that cannot be quickly detected, e.g., the test sample may need to be incubated or otherwise developed over time.

To address that problem, selectivity can be obtained by binding a target biological analyte to, e.g., a detection surface. Selective binding of known target biological analytes to detection surfaces can, however, raise issues when the sensor used relies on acousto-mechanical energy to detect the target biological analyte.

Acoustic wave sensors are so named because their detection mechanism is a mechanical, or acoustic, wave. As the acoustic wave propagates through or on the surface of the material, any changes to the characteristics of the propagation path affect the velocity and/or amplitude of the wave. Changes in velocity can be monitored by measuring the frequency or phase characteristics of the sensor and can then be correlated to the corresponding physical quantity being measured.

Acoustic wave devices are described by the mode of wave propagation through or on a piezoelectric substrate. When the acoustic wave propagates on the surface of the substrate, it is known as a surface wave. The surface acoustic wave sensor (SAW) and the shear-horizontal surface acoustic wave (SH-SAW) sensor are the most widely used surface wave devices. One of the important features of a SH-SAW sensor is that it allows for sensing in liquids.

Shear horizontal surface acoustic wave sensors are designed to propagate a wave of acousto-mechanical energy along the plane of the sensor detection surface. In some systems, a waveguide may be provided at the detection surface to localize the acousto-mechanical wave at the surface and increases the sensitivity of the sensor (as compared to a non-waveguided sensor). This modified shear horizontal surface acoustic wave is often referred to as a Love-wave shear horizontal surface acoustic wave sensor (“LSH-SAW sensor”).

Such sensors have been used in connection with the detection of chemicals and other materials where the size of the target analytes is relatively small. As a result, the mass of the target analytes is largely located within the effective wave field of the sensors (e.g., about 60 nanometers (nm) for a sensor operating at, e.g., a frequency of 103 Megahertz (MHz) in water).

For these sensors, the adsorption of an analyte on the surface perturbs the acoustic waves propagated across the sensor, allowing the detection of an analyte. These perturbations can be measured as changes in the phase and attenuation of the device. In a typical sensing experiment, the sensor is stabilized for some time, the analyte of interest is injected over the sensor and the change in phase and attenuation is measured. The change in phase and/or the change in attenuation is expected to correlate to the presence and possibly the concentration of the target analyte.

The sensors can experience limitations in detection, particularly at lower concentrations of the target analyte in a sample. Several reasons exist for this effect including the fraction of the analyte present that is actually captured on the sensor surface; the mass and/or size of the captured target analyte; and the inherent sensitivity of the SAW device. Thus, a need still exists for improvements in the detection of target analytes using acousto-mechanical detection systems.

### SUMMARY OF THE INVENTION

The present invention provides methods for enhancing the detection of target biological analytes within sample material using acousto-mechanical energy generated by a sensor. The method includes binding the target analytes with magnetic particles and then capturing the target analytes attached to the magnetic particles with the SAW sensor surface. The acousto-mechanical energy may preferably be provided using an acousto-mechanical sensor, e.g., a surface acoustic wave sensor such as a shear horizontal surface acoustic wave sensor (e.g., a LSH-SAW sensor), although other acousto-mechanical sensor technologies may be used in connection with methods of the present invention in some instances. Improvements in the detection limit may be increased as much as a fifty-fold may be achieved using the methods described herein.

In one embodiment, a method of detecting a target biological analyte is provided, the method comprising contacting sample material with magnetic particles, wherein a target biological analyte within the sample material interacts with the magnetic particles such that the target biological analyte is bound to the magnetic particle within the sample material; providing a system comprising an acousto-mechanical device comprising a detection surface with a capture agent located on the detection surface, wherein the capture agent is capable of selectively attaching the target biological analyte to the detection surface; contacting the detection surface of the acousto-mechanical device with the sample material; providing a magnetic field generator capable of providing a magnetic field proximate the detection surface that draws the target analyte with the attached magnetic particles to the sensor surface; selectively attaching the target biological analyte with the attached magnetic particles to the detection surface; disabling the magnetic field generator to substantially reduce the magnetic field proximate the detection surface; and operating the acousto-mechanical device to detect the attached target biological analyte while the detection surface is submersed in liquid.

In another embodiment, a method of detecting a biological analyte, the method comprising fractionating target biological analyte located within sample material; contacting the fractionated target biological analyte with magnetic particles, wherein the fractionated target biological analyte within the sample material interacts with the magnetic particles such that the fractionated target biological analyte is bound to the magnetic particle within the sample material; providing a system comprising a surface acoustic wave device comprising a detection surface with a capture agent located on the detection surface, wherein the capture agent is capable of selectively attaching the target biological analyte to the detection surface; contacting the detection surface of the surface acoustic wave device with the sample material; providing a magnetic field generator capable of providing a magnetic field proximate the detection surface that draws the target analyte attached to the magnetic particles to the sensor surface; selectively attaching the target biological analyte to the detection surface; removing the magnetic field generator a sufficient distance from the detection surface to substantially reduce the magnetic field proximate the detection surface; and operating the surface acoustic wave sensor to detect the attached fractionated target biological analyte while the detection surface is submersed in liquid.

As used herein and in the appended claims, the singular forms “a,” “and,” and “the” include plural referents unless the context clearly dictates otherwise. Thus, for example, reference to “a target biological analyte” includes a plurality of target biological analytes and reference to “the detection chamber” includes reference to one or more detection chambers and equivalents thereof known to those skilled in the art.

The terms “comprises” and variations thereof do not have a limiting meaning where these terms appear in the description or the claims.

These and other features and advantages of the detection systems and methods of the present invention may be described in connection with various illustrative embodiments of the invention below.

### BRIEF DESCRIPTION OF THE DRAWINGS

FIG. 1 is a representation of an acoustic sensor.

FIG. 2 is a schematic diagram of one exemplary detection apparatus including a biosensor.

FIG. 3 is a schematic diagram of a detection apparatus including a biosensor.

FIG. 4 is a schematic diagram of an acoustic sensor detection system.

### DETAILED DESCRIPTION OF EXEMPLARY EMBODIMENTS OF THE INVENTION

In the following detailed description of exemplary embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

The method of the present invention involves capturing the target molecules on the surface of magnetic particles and capturing the magnetic particles on a sensor surface. The use of magnetic particles increases the amount of target analyte that can be delivered to the sensor surface. Further, the coupling of the magnetic particles (via the target analyte) to the sensor surface can enhance the sensor response.

As discussed above, one issue that may be raised in the use of acousto-mechanical energy to detect the presence or absence of target biological analyte in sample material is the ability to effectively couple the target biological analyte to the detection surface such that the acousto-mechanical energy from the sensor is affected in a detectable manner. As used herein, “target biological analyte” may include, e.g., microorganisms (e.g., bacteria, viruses, endospores, fungi, protozoans, etc.), proteins, peptides, amino acids, fatty acids, nucleic acids, carbohydrates, hormones, steroids, lipids, vitamins, etc.

The detection methods of the present invention may, in some embodiments, provide a variety of techniques for detecting the target biological analytes in sample material. The method includes optionally fractionating or disassembling the target biological analytes in the sample material (e.g., lysing the target biological analyte), binding the magnetic particles to the target biological analyte, and contacting the target analyte bound to the magnetic particles with the surface of a SAW sensor. The SAW sensor is coated with a capture agent with an affinity to the target analyte. A magnetic field is applied to the surface of the sensor to draw the target analyte bound to the magnetic particles down to the sensor surface. Once drawn down to the sensor surface, the magnetic field is removed and the sensor response is measured.

The target biological analyte may be obtained from sample material that is or that includes a test specimen obtained by any suitable method and may largely be dependent on the type of target biological agent to be detected. For example, the test specimen may be obtained from a subject (human, animal, etc.) or other source by e.g., collecting a biological tissue and/or fluid sample (e.g., blood, urine, feces, saliva, semen, bile, ocular lens fluid, synovial fluid, cerebral spinal fluid, pus, sweat, exudate, mucous, lactation milk, skin, hair, nails, etc.). In other instances, the test specimen may be obtained as an environmental sample, product sample, food sample, etc. The test specimen as obtained may be a liquid, gas, solid or combination thereof.

Before delivery to the systems and methods of the present invention, the sample material and/or test specimen may be subjected to prior treatment, e.g., dilution of viscous fluids, concentration, filtration, distillation, dialysis, addition of reagents, chemical treatment, etc.

The capture of the target analyte to the magnetic particle is accomplished by using magnetic particles coated with a capture agent with an affinity to the target analyte. The capture agent may bind to the target analyte by specific or non-specific binding. For instance, streptavidin-coated particles such as those available from Invitrogen (Carlsbad, Calif.) and Chemicell GmbH (Berlin, Del.) may be used to capture Protein A-biotin. Similarly, other target analytes can be captured by coating biotinlyated proteins such as a streptavidin-coated magnetic particle with a biotinylated antibody that is specific to the target analyte. In one embodiment, the capture of a target analyte, such as Staphylococcus aureus, comprises contacting the test sample with a streptavidin-coated magnetic particle with a biotinylated derivative of a monoclonal antibody attached thereto (such as the Mab 107 monoclonal antibody described in U.S. patent application Ser. No. 11/562,747, filed Nov. 22, 2006, entitled “ANTIBODY WITH PROTEIN A SELECTIVITY”). The magnetic particles are then injected over the sensor surface and captured by a magnetic field generator that is located proximate the sensor surface.

The capture of the magnetic particle attached to the target analyte on the sensor surface is influenced by several factors including the strength of the magnetic field (as determined by the strength of the magnet and the distance of the magnet from the sensor), the location of the magnetic field generator relative to the sensor surface in the X-Y plane, the orientation of the magnetic field generator, the size and composition of the magnetic particle, and the flow rate of the test sample over the sensor during capture and movement of the magnetic field generator. For instance, one may place the magnetic field generator immediately adjacent the sensor surface (for example, at a zero mm distance) and capture all particles at the leading edge of the sensor for a given flow rate. The magnetic field generator then may be removed to release the cluster of particles to flow over the sensor. Alternately, the magnetic field generator may be positioned some distance away from the sensor while still maintaining a magnetic field that attracts the magnetic particles, and with the use of an appropriate flow rate the particles may be uniformly coated over the sensor in a single step.

While capturing target biological analytes with a coated magnetic particle is known in the prior art, it has not heretofor been recognized that the magnetic field generated to manipulate the magnetic particles to the surface have an adverse impact on the detection ability of the SAW sensor. After sensor the magnetic particles have been captured on the sensor surface, the magnetic field is substantially reduced proximate the sensor surface (e.g., by removal or otherwise disabled) in order for the sensor to respond. There is a sensor response when the magnetic field generator is in place, however it does not correlate to the analyte concentration. On the other hand, after the magnetic field generator is disabled, a large signal is observed that correlates to the analyte concentration. Thus, removing the magnetic field generator post-capture improves the sensitivity of the assay. This is true even in the case where all the particles are coated uniformly over the sensor by optimizing the magnetic field generator position and flow rate.

In preferred embodiments, the magnet is maintained in a consistent location proximate the sensor surface with minimal movement during capture of the magnetic particles. Movement of the magnet, or the sensor surface relative to the magnet, may result in erratic sensor response. Although not intending to be bound by theory, the movement of the magnet relative to the sensor surface can be detected by the sensor, and thus interfere with the sensor's response.

In a preferred embodiment, the removal (or other disablement) and subsequent sensor measurement is performed when the sensor surface is coated at least in part with fluid. In most embodiments the sensor surface is submersed in fluid, e.g., in a liquid sample material containing the magnetic particles with target analyte attached.

In one embodiment utilizing the methods described herein, the detection limit or sensitivity of the sensor for a given target biological analyte, such as Protein A, can be increased as much as a fifty-fold. As further exemplified in the Examples below, the sensor can only detect 50 ng per 500 microliters or greater of target biological analyte in the sample material when the sensor is operated without attachment of the target biological analyte to magnetic particles or application/disablement of the magnetic generator as described and claimed herein. In contrast, utilizing the methods as described and claimed herein, the sensor can detect as low as 1 ng per 500 microliters of target biological analyte in the sample material.

Additionally, the magnetic particles used are preferably less than one micron in size when used with a SH-SAW sensor. If the particles are bigger than one micron, detection may be adversely impacted with a SH-SAW because even with a closely packed coating, there may be a reduced number of point contacts that effect the wave propagation at the sensor surface. In a preferred embodiment, the magnetic particles are 250 nm, and more preferably 100 nm in size.

Traditionally Love mode sensors have shown a response in the phase. For the assay of this invention, we found that there was no meaningful response in phase. The response was instead in the attenuation, after the magnetic field generator has been moved away from the sensor.

The data generated in the experiments is typically gathered in the frequency domain. The data can be transformed into the time domain and a time gating algorithm can be performed. In a typical algorithm, the gates are applied to filter out undesirable time signals, and the data can then be transformed back into the frequency domain. For the case in which the sensor has a reference channel, the reference channel signal can be subtracted from the active channel signal to filter out undesirable noise. The response is typically calculated from the portion of the data after the magnetic field generator is substantially reduced from proximate the sensor surface. The attenuation shift is measured as the difference between the attenuation just before the magnetic field generator is reduced and the attenuation within a few minutes after the magnetic field generator is reduced. If there is drift before the magnetic field generator is reduced, the sensor drift can be estimated using a linear regression on data points before the magnetic field generator removal. The measured shift can then be corrected for this drift.

The use of magnetic particles to bind to the target analyte provides an enhancement in capture efficiency of target analytes on the sensor surface. Significantly higher amounts of target analyte can be delivered to the sensor surface. For example, relying on diffusion alone leads to approximately 0.1 to 1% of the available target analytes reaching the sensor surface. However, with the application of the magnetic field generator to the magnetic particles bound to target analytes, up to 100% delivery and/or retention of the target analyte to the sensor surface is possible. Because the target analytes are bound on the magnetic particles drawn to the sensor surface, the target analytes are moved to the surface at much higher rates than other constituents of the sample. Hence, preferential attachment of the target analyte to the sensor surface is achieved.

### Particle Attachment

The target biological analyte is attached to magnetic particles with selective affinity to the target biological analyte. The particles may be attached in combination with fractionating/disassembly techniques (where, e.g., the particles could attach to fragments of a cell wall, etc.). In some embodiments, the target biological analyte is fractionated or otherwise disassembled into smaller fragments or particles such that an increased percentage of the target biological analyte bound to the magnetic particles can be retained within the effective wave field of the acousto-mechanical sensor and/or effectively coupled with the detection surface of the acousto-mechanical sensor.

The fractionating or disassembly may be accomplished chemically, mechanically, electrically, thermally, or through combinations of two or more such techniques. Examples of some potentially suitable chemical fractionating techniques may involve, e.g., the use of one or more enzymes, hypertonic solutions, hypotonic solutions, detergents, etc. Examples of some potentially suitable mechanical fraction ating techniques may include, e.g., exposure to sonic energy, mechanical agitation (e.g., in the presence of beads or other particles to enhance breakdown), alkaline lysis etc. Thermal fractionating may be performed by, e.g., heating the target biological agent. Other fractionating/disassembly techniques may also be used in connection with the present invention. U.S. Patent Application Publication No. 2005-0153370-A1 titled “Method of Enhancing Signal Detection of Cell-Wall Components of Cells”, filed on Dec. 17, 2004, describes the use of lysing as one method of fractionating a target biological analyte that may be used in connection with the present invention.

In other instances, the magnetic particles may be used in the absence of intentional fractionating/disassembly of the target biological analyte. For example, the magnetic particles can be used for capturing bacterial whole cells, as described in U.S. Patent Application 60/867,016, entitled “Method of Capturing Bacterial Whole Cells,” filed on Nov. 22, 2006, and incorporated by reference in its entirety herein. The particles may selectively attach to the target biological analyte or they may non-selectively attach to materials within a test sample.

Particles attached to the target biological analyte (or fragments thereof) are magnetic such that they can be acted on by a magnetic field applied to the sensor before measuring detection. In such a system, a magnetic field is positioned proximate the detection surface such that the target biological analytes are attracted and attached to the detection surface where they can be detected by the acousto-mechanical sensor.

Magnetic particles enhance detection of the target biological analyte in a number of ways. The magnetic particles are used to drive the attached target biological analyte to the detection surface under the influence of a magnetic field, thus accelerating capture and/or increasing capture efficiency. The attached magnetic particles themselves may also provide enhanced detection when coupled to the sensor surface via the target analyte.

General methods of using magnetic particles and methods of making magnetic particles may be described in, e.g., U.S. Pat. No. 3,970,518 (Giaever); U.S. Pat. No. 4,001,197 (Mitchell et al.); and EP Publication No. 0016552 (Widder et al.). These methods may be adapted for use in connection with the present invention.

Reagents may also be added that cause a change in the viscous, elastic, and/or viscoelastic properties of the sample material in contact with the detection surface. Examples of some suitable mass-modification techniques may be, e.g., the use of fibrinogen in combination with Staphylococcus species as described in, e.g., U.S. Patent Application Ser. No. 60/533,171, filed on Dec. 30, 2003 and U.S. Patent Application Publication No. 2006-0019330-A1.

### Selective Attachment

The detection systems and methods of the present invention may preferably provide for the selective attachment of target biological analyte to the magnetic particles as well as the detection surface or to another component that can be coupled to the detection surface. Selective attachment may be achieved by a variety of techniques. Some examples may include, e.g., antigen-antibody binding; affinity binding (e.g., avidin-biotin, nickel chelates, glutathione-GST); covalent attachment (e.g., azlactone, trichlorotriazine, phosphonitrilic chloride trimer or N-sulfonylaminocarbonyl-amide chemistries); etc.

The selective attachment of a target biological analyte may be achieved directly, i.e., the target biological analyte itself is selectively attached to the detection surface. Examples of some such direct selective attachment techniques may include the use of capture agents, e.g., antigen-antibody binding (where the target biological analyte itself includes the antigen bound to an antibody immobilized on the detection surface), DNA capture, etc.

The selective attachment may also be indirect, i.e., where the target biological analyte is selectively attached to the magnetic particle that is selectively attached or attracted to the detection surface. The indirect selective attachment technique includes selectively binding magnetic particles to the target biological analyte such that the target biological analyte can be magnetically attracted to and retained on the detection surface.

In connection with selective attachment, it may be preferred that systems and methods of the present invention provide for low non-specific binding of other biological analytes or materials to, e.g., the detection surface. Non-specific binding can adversely affect the accuracy of results obtained using the detection systems and methods of the present invention. Non-specific binding can be addressed in many different manners. For example, biological analytes and materials that are known to potentially raise non-specific binding issues may be removed from the test sample before it is introduced to the detection surface. In another approach, blocking techniques may be used to reduce non-specific binding on the detection surface.

Because selective attachment may often rely on coatings, layers, etc. located on the acousto-mechanical detection surface, care must be taken that these materials are relatively thin and do not dampen the acousto-mechanical energy to such a degree that effective detection is prevented.

Another issue associated with selective attachment is the use of what are commonly referred to as “immobilization” technologies that may be used to hold or immobilize a capture agent on the surface of, e.g., the acousto-mechanical sensor itself. The detection systems and methods of the present invention may involve the use of a variety of immobilization technologies.

As discussed herein, the general approach is to coat or otherwise provide the detection surface of an acousto-mechanical sensor device with capture agents such as, e.g., antibodies, peptides, aptamers, or any other capture agent that has affinity for the target biological analyte. The surface coverage and packing of the capture agent on the surface may determine the sensitivity of the sensor. The immobilization chemistry that links the capture agent to the detection surface of the sensor may play a role in the packing of the capture agents, preserving the activity of the capture agent (if it is a biomolecule), and may also contribute to the reproducibility and shelf-life of the sensor.

If the capture agents are proteins or antibodies, they can nonspecifically adsorb to a surface and lose their ability (activity) to capture the target biological analyte. A variety of immobilization methods may be used in connection with acousto-mechanical sensors to achieve the goals of high yield, activity, shelf-life and stability. These capture agents may preferably be coated using glutaraldehyde cross-linking chemistries, entrapment in acrylamide, or general coupling chemistries like carbodiimide, epoxides, cyano bromides etc. Depending on the capture agent used, the concentration of capture agent on the sensor surface may become important in optimizing the sensor response.

Apart from the chemistry that binds to the capture agent and still keeps it active, there are other surface characteristics of any immobilization chemistries used in connection with the present invention that may need to be considered and that may become relevant in an acousto-mechanical sensor application. For example, the immobilization chemistries may preferably cause limited damping of the acousto-mechanical energy such that the immobilization chemistry does not prevent the sensor from yielding usable data. The immobilization chemistry may also determine how the antibody or protein is bound to the surface and, hence, the orientation of the active site of capture. The immobilization chemistry may preferably provide reproducible characteristics to obtain reproducible data and sensitivity from the acousto-mechanical sensors of the present invention.

Some immobilization technologies that may be used in connection with the systems and methods of the present invention may be described in, e.g., U.S. Patent Application Publication Nos. 2005-01070615-A1; and 2005-0112672-A1 and U.S. Patent Ser. Nos. 60/533,162, filed on Dec. 30, 2003; 60/533,178, filed on Dec. 30, 2003, U.S. Patent Application Publication Nos. 2005-0142296-A1; 2005-0106709-A1; 2005-0227076-A1; 2006-0135718-A1; 2006-0135783-A1; and PCT Publication No. WO2005/066092 titled “Acoustic Sensors and Methods”, filed on Dec. 17, 2004.

Immobilization approaches may include a tie layer between the waveguide on an acousto-mechanical substrate and the immobilization layer. One exemplary tie layer may be, e.g., a layer of diamond-like glass, such as described in International Publication No. WO 01/66820 A1 (David et al.).

### Acousto-Mechanical Sensors

The systems and methods of the present invention preferably detect the presence of target biological analyte in a test sample through the use of acousto-mechanical energy that is measured or otherwise sensed to determine wave attenuation, phase changes, frequency changes, and/or resonant frequency changes.

The acousto-mechanical energy may be generated using, e.g., piezoelectric-based surface acoustic wave (SAW) devices. As described in, e.g., U.S. Pat. No. 5,814,525 (Renschler et al.), the class of piezoelectric-based acoustic mechanical devices can be further subdivided into surface acoustic wave (SAW), acoustic plate mode (APM), or quartz crystal microbalance (QCM) devices depending on their mode of detection.

The methods described herein employ an acoustic sensor, and more specifically, an acoustic mechanical biosensor, that detects a change in at least one physical property and produces a signal in response to the detectable change. Preferably, the acoustic mechanical biosensor employed herein is a surface acoustic wave (SAW) biosensor. In these devices an acoustic wave is generated from an interdigitated transducer (IDT) on a piezoelectric substrate either as a surface acoustic wave or as a bulk acoustic wave. A second IDT may convert the acoustic wave back to an electric signal for measurement. This is referred to as a delay line. Alternatively the device may operate as a resonator. The space between the two IDTs can be modified with a coating that may include reactive molecules for chemical or biosensing applications.

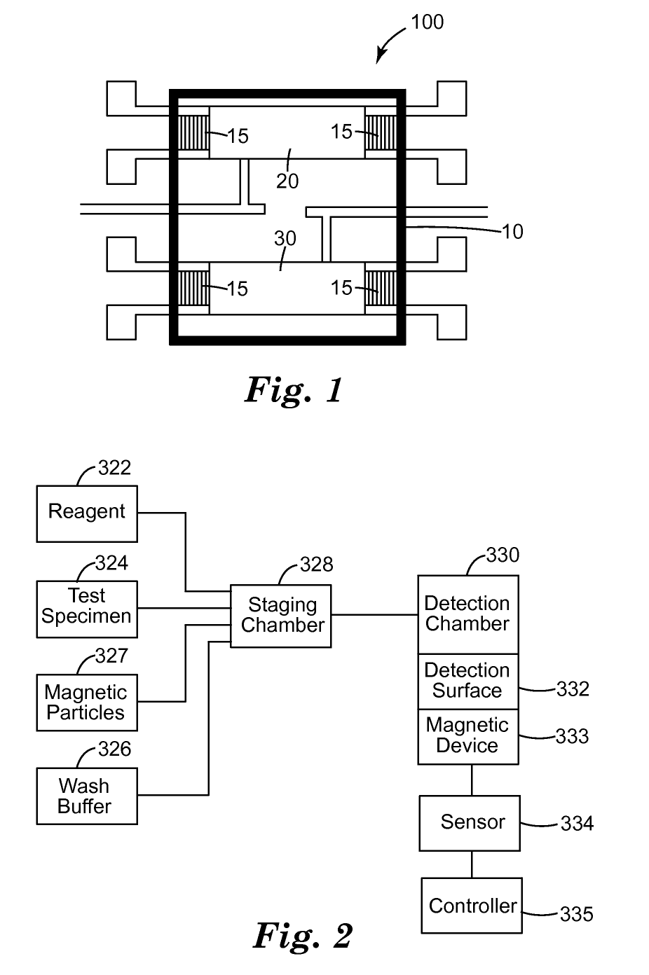

With reference to FIG. 1, in some embodiments the acoustic mechanical biosensor surface 100 between the IDTs 15 preferably comprises two delay lines. A first channel, i.e. the “active” channel 20 is provided for receipt of the test sample. The second channel, i.e. the “reference” channel 30 is provided as the baseline or control. Accordingly, the change in physical property is the difference between the active channel and the reference channel. When necessary, an acoustic waveguide 10 (only the boundaries of which are depicted in FIG. 1) typically covers the area between the IDTs as well as the IDTs themselves. The data may be transformed with mathematical algorithms in order to improve the sensitivity. Alternative configurations of an exemplary acoustic mechanical sensor include those disclosed in PCT Publication No. WO2005/075973 titled “Acousto-mechanical Detection Systems and Methods of Use”, filed Dec. 17, 2004.

Piezoelectric-based SAW biosensors typically operate on the basis of their ability to detect minute changes in mass or viscosity. As described in U.S. Pat. No. 5,814,525, the class of piezoelectric-based acoustic mechanical biosensors can be further subdivided into surface acoustic wave (SAW), acoustic plate mode (APM), or quartz crystal microbalance (QCM) devices depending on their mode of detection of mass changes.

In some embodiments, the acoustic mechanical biosensor includes a secondary capture agent or reactant (e.g., antibody) that attaches the target analyte to the surface of the piezoelectric acoustic mechanical biosensor. The propagation velocity of the surface wave is a sensitive probe capable of detecting changes such as mass, elasticity, viscoelasticity, conductivity and dielectric constant. Thus, changes in any of these properties results in a detectable change in the surface acoustic wave. That is, when a substance comes in contacts with, absorbs, or is otherwise caused to adhere to the surface coating of a SAW device, a corresponding response is produced.

APM can also be operated with the device in contact with a liquid. Similarly, an alternating voltage applied to the two opposite electrodes on a QCM (typically AT-cut quartz) device induces a thickness shear wave mode whose resonance frequency changes in proportion to mass changes in a coating material.

The direction of the acoustic wave propagation (e.g., in the plane parallel to the waveguide or perpendicular to the plane of the waveguide) is determined by the crystal-cut of the piezoelectric material from which the acoustic mechanical biosensor is constructed. SAW biosensors that have the majority of the acoustic wave propagating in and out of the plane (i.e., Rayleigh wave, most Lamb-waves) are typically not employed in liquid sensing applications since there is too much acoustic damping from the liquid contact with the surface.

For liquid sample mediums, a shear horizontal surface acoustic wave biosensor (SH-SAW) is preferably constructed from a piezoelectric material with a crystal-cut and orientation that allows the wave propagation to be rotated to a shear horizontal mode, i.e., in plane of the biosensor waveguide), resulting in reduced acoustic damping loss to the liquid in contact with the biosensor surface. Shear horizontal acoustic waves include, e.g., acoustic plate modes (APM), surface skimming bulk waves (SSBW), Love-waves, leaky acoustic waves (LSAW), and Bleustein-Gulyaev (BG) waves.

In particular, Love mode sensors consist of a substrate supporting a SH wave mode such as SSBW of ST quartz or the leaky wave of 36°YXLiTaO₃. These modes are converted into a Love-wave mode by application of thin acoustic guiding layer or waveguide. These waves are frequency dependent and can be generated provided that the shear wave velocity of the waveguide layer is lower than that of the piezoelectric substrate. SiO₂ has been used as an acoustic waveguide layer on quartz. Other thermoplastic and crosslinked polymeric waveguide materials such as polymethylmethacrylate, phenol-formaldehyde resin (e.g., trade designation NOVALAC), polyimide and polystyrene, have also been employed.

Alternatively QCM devices can also be used with liquid sample mediums, although with these devices the acoustic wave will be severely damped by the liquid medium, leading to a generally less sensitive device.

Biosensors employing acoustic mechanical means and components of such biosensors are known. See, for example, U.S. Pat. Nos. 5,076,094; 5,117,146; 5,235,235; 5,151,110; 5,763,283; 5,814,525; 5,836,203; 6,232,139. SH-SAW devices can be obtained from various manufacturers such as Sandia National Laboratories, Albuquerque, N. Mex. Certain SH-SAW biosensors are also described in “Low-level detection of a Bacillus anthracis stimulant using Love-wave biosensors of 36° YXLiTaO₃,” Biosensors and Bioelectronics, 19, 849-859 (2004). SAW biosensors, as well as methods of detecting biological agents, are also described in U.S. Patent Application Ser. No. 60/533,169, filed Dec. 30, 2003.

In some embodiments, the surface of the biosensor includes a secondary capture agent or reactant (e.g., antibody) overlying the waveguide layer. In this embodiment, the biosensor typically detects a change in viscosity and/or mass bound by the secondary capture agent or reactant. For this embodiment, the biosensor preferably includes an immobilization layer (overlying the waveguide layer) and optional tie layer(s).

An immobilization layer can be provided for the purpose of binding the secondary capture agent or reactant (e.g., antibody) to the surface. Materials useful for the immobilization layer include those described above.

### Detection Systems and Cartridges

As discussed herein, the materials and methods of the present invention may be used on sensors to provide waveguides, immobilization layers, capture materials, or combinations thereof. The following discussion presents some potential examples of systems and detection cartridges in which the sensors using the materials of the present invention may be used.

FIG. 2 is a schematic diagram of one detection apparatus including a biosensor. The depicted apparatus may optionally include a reagent 322, test specimen 324, wash buffer 326, and magnetic particles 327. These various components may be introduced into, e.g., a staging chamber 328 where they may intermix and/or react with each other. Alternatively, one or more these components may be present in the staging chamber 328 before one or more of the other components are introduced therein.

For example, it may be desirable that the reagent 322 and the test specimen 324 be introduced into the staging chamber 328 to allow the reagent 322 to act on and/or attach to the target biological analyte within the test specimen 324. Following that, the magnetic particles 327 may be introduced into the staging chamber 328. The magnetic particles 327 may selectively attach to the target biological analyte material within the staging chamber 328, although they do not necessarily need to do so.

After attachment of the target biological analyte in the test specimen 324 to the magnetic particles 327, the test specimen 324 (and associated magnetic particles) may be moved from the staging chamber 328 to the detection chamber 330 where the target biological analyte in the sample material can contact the detection surface 332 of a sensor. The detection surface 332 may preferably be of the type that includes capture agents located thereon such that the target biological analyte in the sample material is selectively attached to the detection surface 332.

If the target biological analyte is associated with magnetic particles, it may be desirable to include a magnetic device 333 capable of generating a magnetic field at the detection surface 332 such that the target biological analyte associated with magnetic particles can be magnetically drawn towards the detection surface for detection using sensor 334 operated by controller 335. The use of magnetic particles in connection with the target biological analyte may enhance detection by, e.g., moving the target biological analyte to the detection surface 332 more rapidly than might be expected in the absence of, e.g., magnetic attractive forces.

It may be preferred that the reagent 322 be selective to the target biological analyte, i.e., that other biological analytes in the test specimen 324 are not modified by the reagent 322. Alternatively, the reagent 322 may be non-selective, i.e., it may act on a number of biological analytes in the test specimen 324, regardless of whether the biological analytes are the target biological analyte or not. In some embodiments, the reagent 322 may preferably be a chemical fractionating agent such as, e.g., one or more enzymes, hypertonic solutions, hypotonic solutions, detergents, etc.

The attachment of biological analytes to, e.g., magnetic particles, may be described generally in, e.g., International Publication Nos. WO 02/090565 (Ritterband) and WO 00/70040 (Bitner et al.) which describe the use of magnetic beads in kits to concentrate cells, as well as magnetically responsive particles. Selective attachment of a biological agent to magnetic particles (e.g., paramagnetic microspheres) is also described in, e.g., Kim et al., “Impedance characterization of a piezoelectric immunosensor part II: Salmonella typhimurium detection using magnetic enhancement,” Biosensors and Bioelectronics 18 (2003) 91-99.

After attachment of the target biological analyte in the test specimen 324 to the magnetic particles 327, the sample material (with the test specimen 324 and associated magnetic particles) may be moved from the staging chamber 328 to the detection chamber 330 where the target biological analyte in the sample material can contact the detection surface 332. Because the target biological analyte is associated with magnetic particles, it may be desirable to include a magnetic field generator 333 capable of generating a magnetic field at the detection surface 332 such that the target biological analyte associated with magnetic particles can be retained on the detection surface for subsequent detection using sensor 334 operated by controller 335. In other words, the magnetic forces provided by the magnetic field proximate the detection surface 332 may draw the magnetic particles (and attached target biological analyte) to the detection surface 332. The magnetic field generator 333 may be any suitable device that can provide a magnetic field arranged to draw magnetic particles to the detection surface, e.g., a permanent magnet, electromagnet, etc.

The use of magnetic particles in connection with the target biological analyte may enhance detection by, e.g., moving the target biological analyte to the detection surface 332 more efficiently and/or rapidly than might be expected in the absence of, e.g., magnetic attractive forces.

Before detection of target analytes with the sensor 334, magnetic field generator 333 is removed from sensor 334 a sufficient distance (or otherwise disabled such as turning off the magnetic field generator 333) to significantly reduce the magnetic field proximate the sensor 334. If the magnetic field is maintained during the detection process (when acoustic energy is being generated and detected) by sensor 334, the magnetic field will negatively impact and most likely prevent accurate detection of the target biological analyte.

If the detection surface 332 includes selective capture agents located thereon such that the target biological analyte is selectively attached to the detection surface 332 once the magnetic field is removed, then the magnetic particles that are not carrying (or being carried by) any target biological analyte may be removed from the detection surface 332 by, e.g., removing the magnetic field. In a SH-SAW sensor, washing the detection surface 332 to remove magnetic particles that are not carrying (or being carried by) target biological analytes is not critical due to the localized detection zone of the SH-SAW. Other methods of removing non-associated magnetic particles, i.e., magnetic particles that are not associated with any target biological analyte, may be performed before introducing the associated magnetic particles (i.e., magnetic particles carrying or being carried by target biological analyte).

Detection of any target biological analytes selectively attached to the detection surface preferably occurs using the sensor 334 as operated by an optional control module 335. The control module 335 may preferably operate the sensor 334 such that the appropriate acousto-mechanical energy is generated. The control module 335 may optionally also set the appropriate flow rate, control movement of the magnetic field generator 333, and also monitor the sensor 334 such that a determination of the presence or absence of the target biological analyte on the detection surface 332 can be made.

Examples of techniques for driving and monitoring acousto-mechanical sensors such as those that may be used in connection with the present invention may be found in, e.g., U.S. Pat. No. 5,076,094 (Frye et al.); U.S. Pat. No. 5,117,146 (Martin et al.); U.S. Pat. No. 5,235,235 (Martin et al.); U.S. Pat. No. 5,151,110 (Bein et al.); U.S. Pat. No. 5,763,283 (Cernosek et al.); U.S. Pat. No. 5,814,525 (Renschler et al.); U.S. Pat. No. 5,836,203 ((Martin et al.); and U.S. Pat. No. 6,232,139 (Casalnuovo et al.), etc. Further examples may be described in, e.g., Branch et al., “Low-level detection of a Bacillus anthracis simulant using Love-wave biosensors on 36°YX LiTaO₃,” Biosensors and Bioelectronics, 19, 849-859 (2004); as well as in U.S. Patent Application No. 60/533,177, filed on Dec. 30, 2003, and PCT Publication No. WO 2005/066622, titled “Estimating Propagation Velocity Through A Surface Acoustic Wave Sensor”, filed on Dec. 17, 2004.

Although an exemplary detection apparatus that may be used in connection with the present invention is discussed above in connection with FIG. 2, those apparatus may be contained in an integrated unit that may be described as a detection cartridge. Exemplary detection cartridges are further described in PCT Publication No. WO2005/075973 titled “Acousto-mechanical Detection Systems and Methods of Use”, filed Dec. 17, 2004 and PCT Publication No. WO2005/064349, titled “Detection Cartridges, Modules, Systems and Methods”, filed on Dec. 17, 2004, which describe additional features of detection cartridges and/or modules that may be used in connection with the present invention.

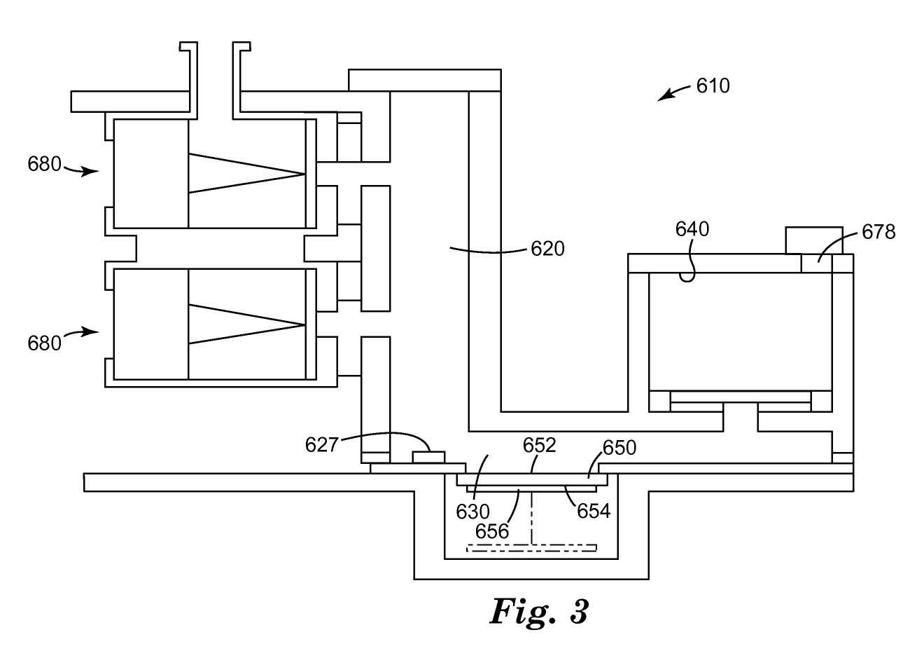

One exemplary embodiment of a detection cartridge 610 including a staging chamber 620, detection chamber 630 and waste chamber 640 is depicted in FIG. 3. The detection cartridge 610 includes a sensor 650 having a detection surface 652 exposed within the detection chamber 630, and a magnetic field generator 656.

It may be preferred that the sensor 650 be an acousto-mechanical sensor such as, e.g., a Love mode shear horizontal surface acoustic wave sensor. As depicted, the sensor 650 may preferably be attached such that, with the possible exception of the magnetic field generator 656, the backside 654 of the sensor 650 (i.e., the surface facing away from the detection chamber 630) does not contact any other structures within the cartridge 610.

In a preferred embodiment shown in FIG. 3, the magnetic field generator 656 is preferably moved proximate the sensor 650 through an opening in cartridge 610. In alternate embodiments, the magnetic field generator 656 may be placed proximate the sensor 650 without an opening, depending on the strength of the magnetic field generator 656, the material used to construct the cartridge 610, etc.

Examples of some potentially suitable methods of attaching acousto-mechanical sensors within a cartridge that may be used in connection with the present invention may be found in, e.g., U.S. Patent Application Ser. No. 60/533,176, filed on Dec. 30, 2003 as well as PCT Publication No. WO 2005/066621, titled “Surface Acoustic Wave Sensor Assemblies”, filed on Dec. 17, 2004.

In some instances, the processes used in the above-identified documents may be used with acoustic sensors that include contact pads that are exposed outside of the boundaries of a waveguide layer on the sensor using a Z-axis adhesive interposed between the sensor contact pads and traces on a carrier or support element to which the sensor is attached. Alternatively, however, the methods described in those documents may be used to make electrical connections through a waveguide layer where the properties (e.g., glass transition point (T_{g}) and melting point) of the Z-axis adhesive and the waveguide material are similar. In such a process, the waveguide material need not be removed from the contact pads on the sensor, with the conductive particles in the Z-axis adhesive making electrical contact through the waveguide material on the contact pads of the sensor.

The embodiment of FIG. 3 includes a vent 678 in the waste chamber 640 that may place the interior volume of the waste chamber 640 in communication with ambient atmosphere. Opening and/or closing the vent 678 may be used to control fluid flow into the waste chamber 640 and, thus, through the cartridge 610. Furthermore, the vent 678 may be used to reduce pressure within the waste chamber 640 by, e.g., drawing a vacuum, etc. through the vent 678.

Although depicted as being in direct fluid communication with the waste chamber 640, one or more vents may be provided and they may be directly connected to any suitable location that leads to the interior volume of the detection cartridge 610, e.g., staging chamber 620, detection chamber 630, etc. The vent 678 may take any suitable form, e.g., one or more voids, tubes, fitting, etc.

Referring again to the cartridge depicted in FIG. 3, the staging chamber 620 may be provided upstream from the detection chamber 630. The staging chamber 620 may provide a volume into which various components may be introduced before entering the detection chamber 630. Although not depicted, it should be understood that the staging chamber 620 could include a variety of features such as, e.g., one or more reagents located therein (e.g., dried down or otherwise contained for selective release at an appropriate time); coatings (e.g., hydrophilic, hydrophobic, etc.); structures/shapes (that may, e.g., reduce/prevent bubble formation, improve/cause mixing, etc.).

Also, the fluid path between the staging chamber 620 and the detection chamber 630 may be open as depicted in FIG. 3. Alternatively, the fluid path between the staging chamber 620 and the detection chamber 630 may include a variety features that may perform one or more functions such as, e.g., filtration (using, e.g., porous membranes, size exclusion structures, beads, etc.), flow control (using, e.g., one or more valves, porous membranes, capillary tubes or channels, flow restrictors, etc.), coatings (e.g., hydrophilic, hydrophobic, etc.), structures/shapes (that may, e.g., reduce/prevent bubble formation and/or transfer, improve mixing, etc.).

Other optional features of the sensor cartridge, such as fluid monitors 627 and modules 680 for delivering various materials are further described in PCT Publication No. WO2005/075973.

Although the exemplary embodiments described herein may include a single sensor, the detection cartridges of the present invention may include two or more sensors, with the two or more sensors being substantially similar to each other or different. Furthermore, each sensor in a detection cartridge according to the present invention may include two or more channels (e.g., a detection channel and a reference channel). Alternatively, different sensors may be used to provide a detection channel and a reference channel within a detection cartridge. If multiple sensors are provided, they may be located in the same detection chamber or in different detection chambers within a detection cartridge.

Additional discussion related to various detection systems and components (such as detection cartridges including biosensors) may be found in, e.g., U.S. Patent Application No. 60/533,169, filed Dec. 30, 2003; PCT Publication No. WO2005/075973 titled “Acousto-mechanical Detection Systems and Methods of Use”, filed Dec. 17, 2004 and PCT Publication No. WO2005/064349, titled “Detection Cartridges, Modules, Systems and Methods”, filed on Dec. 17, 2004.

### System Design

It may desirable that the detection cartridges of the present invention be capable of docking with or being connected to a unit that may, e.g., provide a variety of functions such as providing power to the sensors or other devices in the detection cartridge, accepting data generated by the sensor, providing the ability to take user input to control fluid flow and/or sensor operation, etc.

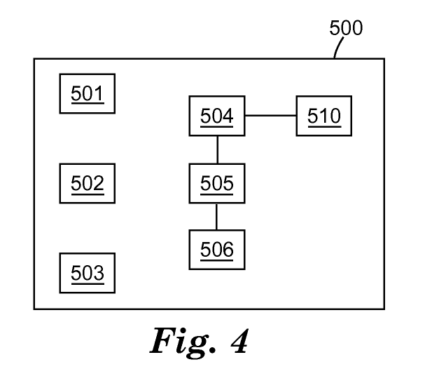

One such system 500 is schematically depicted in FIG. 4, and may preferably include a power source 501 and user interface 502 (e.g., pushbuttons, keyboard, touchscreen, microphone, etc.). The system 500 may also include an identification module 503 adapted to identify a particular detection cartridge 510 using, e.g., barcodes, radio-frequency identification devices, mechanical structures, etc.

The system 500 may also preferably include a sensor analyzer 504 that obtains data from a sensor in the detection cartridge and a processor 505 to interpret the output of the sensor. In other words, sensor analyzer 504 may receive output from a sensor detection cartridge 510 and provide input to processor 505 so that the output of the sensor can be interpreted.

Processor 505 receives input from sensor analyzer 504, which may include, e.g., measurements associated with wave propagation through or over an acousto-mechanical sensor. Processor 505 may then determine whether a target biological analyte is present in sample material. Although the invention is not limited in this respect, the sensor in detection cartridge 510 may be electrically coupled to sensor analyzer 504 via insertion of the detection cartridge 510 into a slot or other docking structure in or on system 500. Processor 505 may be housed in the same unit as sensor analyzer 504 or may be part of a separate unit or separate computer.

Processor 505 may also be coupled to memory 506, which can store one or more different data analysis techniques. Alternatively, any desired data analysis techniques may be designed as, e.g., hardware, within processor 505. In any case, processor 505 executes the data analysis technique to determine whether a detectable amount of a target biological analyte is present on the detection surface of a sensor in detection cartridge 510.

By way of example, processor 505 may be a general-purpose microprocessor that executes software stored in memory 506. In that case, processor 505 may be housed in a specifically designed computer, a general purpose personal computer, workstation, handheld computer, laptop computer, or the like. Alternatively, processor 505 may be an application specific integrated circuit (ASIC) or other specifically designed processor. In any case, processor 505 preferably executes any desired data analysis technique or techniques to determine whether a target biological analyte is present within a test sample.

Memory 506 is one example of a computer readable medium that stores processor executable software instructions that can be applied by processor 505. By way of example, memory 506 may be random access memory (RAM), read-only memory (ROM), non-volatile random access memory (NVRAM), electrically erasable programmable read-only memory (EEPROM), flash memory, or the like. Any data analysis techniques may form part of a larger software program used for analysis of the output of a sensor (e.g., LABVIEW software from National Instruments Corporation, Austin, Tex.).

Still other potentially useful data analysis techniques may be described in the documents identified herein relating to the use of acoustic sensors. Although systems and methods related to the use of surface acoustic wave sensors are described therein, it should be understood that the use of these systems and methods may be used with other acousto-mechanical sensors as well.

### Manufacturing Acousto-Mechanical Sensors

As discussed herein, the present invention relies on the use of acousto-mechanical sensors to detect the presence of target biological analyte within a test sample flowed over a detection surface. Coating or otherwise providing the various materials needed to provide acousto-mechanical sensors with the desired selective attachment properties may be performed using a variety of methods and techniques.

As used with acoustic sensors, the waveguide materials, immobilization materials, capture agents, etc. used on the sensors may be deposited by any suitable technique or method. Typically, it may be preferred that such materials be delivered to a substrate in a carrier liquid, with the carrier liquid and the materials forming, e.g., a solution or dispersion. When so delivered, examples of some suitable deposition techniques for depositing the materials on a surface may include, but are not limited to, flood coating, spin coating, printing, non-contact depositing (e.g., ink jetting, spray jetting, etc.), pattern coating, knife coating, etc. It may be preferred, in some embodiments, that the deposition technique have the capability of pattern coating a surface, i.e., depositing the materials on only selected portions of a surface. U.S. patent application Ser. No. 10/607,698, filed Jun. 27, 2003, describes methods of pattern coating that may be suitable for use in connection with the construction of sensors according to the present invention.

In some embodiments, (such as those described in, e.g., PCT Publication No. WO2005/066092 titled “Acoustic Sensors and Methods”, filed on Dec. 17, 2004 and others), some materials may function as both waveguide material and immobilization material for secondary capture agents on an underlying substrate. In other embodiments, the same materials may function as waveguide material, immobilization material, and capturing material. In both of these variations, the materials of the present invention may preferably be deposited on an underlying substrate that is, itself, effectively insoluble in the carrier liquid such that the carrier liquid does not adversely affect the underlying substrate.

If, however, the surface on which the waveguide materials, immobilization materials, and/or capture agents are to be deposited exhibits some solubility in the carrier liquid used to deliver the material, it may be preferred that the material be deposited using a non-contact deposition technique such as, e.g., ink jetting, spray jetting etc. For example, if the underlying substrate is a waveguide formed of, e.g., polyimide, acrylate, etc., on a sensor substrate and the material of an immobilization layer is to be deposited using, e.g., butyl acetate, as the carrier liquid, then it may be preferred to use a non-contact deposition method to limit deformation of the waveguide and to preferably retain the functional characteristics of the immobilization material exposed on the resulting coated surface. The same considerations may apply to the coating of capture agents on a surface.

There are several variables that may be controlled in a spray-jet coating process, including deposition rate, substrate speed (relative to the spray jet head), sheath gas flow rate, sheath gas, raster spacing, raster pattern, number of passes, percent solids in the sprayed solution/dispersion, nozzle diameter, the carrier liquid, the composition of the underlying surface on which the materials of the present invention are being deposited, etc. Specific conditions under which the materials of the present invention can be deposited to yield a suitable coating may be determined empirically.

The methods of the present invention may be utilized in combination with various materials, methods, systems, apparatus, etc. as described in various U.S. patent applications identified below, all of which are incorporated by reference in their respective entireties. They include U.S. Patent Application Ser. Nos. 60/533,162, filed on Dec. 30, 2003; 60/533,178, filed on Dec. 30, 2003; U.S. Patent Application Publication Nos. 2005-0142296-A1; 2005-0107615-A1; 2005-0112672-A1; 2005-0106709-A1; 2005-0227076-A1; U.S. Patent Application Ser. No. 60/533,171, filed Dec. 30, 2003; U.S. Patent Application Publication No. 2006-0019330-A1; U.S. Patent Application Ser. Nos. 60/533,177, filed Dec. 30, 2003; 60/533,176, filed Dec. 30, 2003; U.S. Patent Application Publication Nos. 2005-0153370-A1; 2006-0135718-A1; 2006-0135783-A1; PCT Publication No. WO 2005/066622, titled “Estimating Propagation Velocity Through A Surface Acoustic Wave Sensor”, filed on Dec. 17, 2004; PCT Publication No. WO 2005/066621, titled “Surface Acoustic Wave Sensor Assemblies”, filed on Dec. 17, 2004; PCT Publication No. WO2005/075973 titled “Acousto-mechanical Detection Systems and Methods of Use”, filed Dec. 17, 2004; PCT Publication No. WO2005/064349, titled “Detection Cartridges, Modules, Systems and Methods”, filed on Dec. 17, 2004; and PCT Publication No. WO2005/066092 titled “Acoustic Sensors and Methods”, filed on Dec. 17, 2004.

### EXAMPLES

These examples are merely for illustrative purposes only and are not meant to be limiting on the scope of the appended claims. All parts, percentages, ratios, etc. in the examples and the rest of the specification are by weight, unless noted otherwise.

### Example 1

### Methods of Preparing Sensors and Running Detection Experiments

A shear-horizontal surface acoustic wave (SH-SAW) sensor (supplied by Com Dev (Cambridge, Ontario, Canada) or by Sandia National Laboratory (Albuquerque, N. Mex.)) spin coated with a waveguide (50:50 copolymer of methyl methacrylate and isobornyl methacrylate prepared as described in Example W1 of PCT Publication No. WO2005/066092 titled “Acoustic Sensors and Methods”, filed on Dec. 17, 2004) was used in the experiments. The sensors were sprayjet-coated with an immobilization chemistry comprising a terpolymer of iso-bornyl methacrylate/methyl methacrylate/Saccharin-methacrylate/acryloyloxybenzophenone 35/35/30/0.5 made in Butyl acetate/Acetonitrile 50/50 prepared as described in Example MP26 of PCT Publication No. WO2005/066092 titled “Acoustic Sensors and Methods”, filed on Dec. 17, 2004. In some cases, a monoclonal antibody (Mab107) specific to Protein A was hand coated or sprayjet-coated on both (active and reference) sensor channels. In other cases, the Mab 107 antibody or the Rabbit anti-staph aureus (RaSa) (Accurate Chemical & Scientific Corporation, Westbury, N.Y.) was hand-coated or sprayjet-coated on one sensor channel (Active channel) and a non-specific Chicken IgY (Jackson ImmunoResearch Laboratories Inc, West Grove, Pa.) was hand-coated or sprayjet-coated on the other channel (Reference channel). The coated sensor was heat-bonded to a flexible circuit via conductive adhesive (Anisotropic conductive film adhesive 7313, 3M Company, St. Paul, Minn.). The bonded sensor was attached to a temperature-controlled flowpod via double-sided adhesive film. The sensor was then connected to an electronic measurement board (via the flex circuit) driven by a software program that was written in LabVIEW software using a network analyzer. LabVIEW software was obtained from National Instruments (Austin, Tex.). Attenuation and phase properties were collected throughout the experiment in the desired frequency range.

To start the experiment, PBSL running buffer (described below) was flowed over the sensor at an average flow rate of 0.1 ml/min via a syringe pump and then adjusted to the desired flow rate. The software program was then used to initiate the experiment. A rare earth magnet composed of Neodymium-Iron-Boron (NdFeB) was raised into position underneath the sensor. After sufficient flow stabilization, the sample was injected via an injection valve and flowed over the sensor at a time specified by the software.

After the sample had reached and collected on the sensor surface, the magnet was moved (“dropped”) a sufficient distance (at a time specified by the software) to significantly reduce the magnetic field strength at the sensor surface. Typically the magnet was moved >65 mm. However, the field strength is significantly reduced at much smaller distances. We observed visually that the magnetic particles were not held (against the force of the bulk liquid flow) at the sensor surface when the magnet was moved to distances >5 mm from the sensor surface.

Flow was continued a sufficient time until the phase and attenuation signals were stabilized. Typically, this was determined by visual inspection of the phase and attenuation raw signals that were displayed on the computer screen. When the changes in the raw signal over time were relatively small compared to the signal changes expected after the magnet was dropped, the signals were considered to be stable.

A time gating algorithm (Page 3-35 and 3-36 in 8753ET/ES Network Analyzers User's Guide, Agilent Technologies) was used to process the raw phase and attenuation data generated from the experiment. Unless specified otherwise, the time interval unit for data collection was 13 seconds and the time commenced when the data collection was started by the software (e.g., time point 100 occurred 1300 seconds after the experiment was started). Appropriate gates for the algorithm may be specified based on the specific sensor design that is being used. The algorithm can be applied directly through the network analyzer such that the data obtained from the experiment is already time gated. Alternatively, the raw data can be collected and time gating can be done using a software program written in Matlab (The Mathworks, Natick, Mass.).

The time gated data were further analyzed to determine shifts in phase and attenuation. All of this data processing was done using the Matlab software. For those cases where there was no reference channel, the shift in the signal for both phase and attenuation in the two channels was computed by subtracting its value just before the magnet is dropped from its value when the signal had stabilized after the magnet was dropped.

For sensors with both an active and reference channel, a difference signal was calculated by subtracting the attenuation and phase signal of the reference channel from that of the active channel, respectively. The shift in this difference signal was computed by subtracting the value just before the magnet is dropped from the stable signal obtained after the magnet was dropped

### Example 2

### Conjugation of Protein A-Biotin to Magnetic Particles

Biotin-conjugated Protein A was obtained from Sigma Chemical Company (St. Louis, Mo.). Streptavidin-coated magnetic particles, obtained from either Invitrogen (Carlsbad, Calif.) or Chemicell Gmbh (Berlin, Germany), were pre-washed in 1 ml Phosphate-buffered Saline (PBS, 137 mM NaCl, 2.7 mM KCl, and 10 mM phosphate, pH 7.40). The magnetic particles and the Protein A-biotin test sample were mixed together at the desired concentrations in 1 ml PBS. The suspension was incubated at 37° C. for ≧30 min (with agitation). The sample was then washed three times in 1 ml PBS to remove any unbound target. The washing process consisted of placing a magnet against the sample tube to immobilize the magnetic particles against the wall of the tube, removing the supernatant, adding an equal volume of fresh PBS and resuspending the particles. For the final wash, the particles were resuspended in 1 ml PBS L64 buffer (PBS buffer containing 0.2% w/v PLURONIC L64 (BASF, Florham Park, N.J.)).

### Example 3

### Conjugation of Protein A Through the Mab107-biotin to Magnetic Particles

Mab 107 was biotinylated using the EZ-Link NHS-PEO4-Biotin kit (Pierce, Rockford, Ill.) according to the manufacturer's instructions. Protein A was obtained from Invitrogen and was diluted in PBS to the desired test concentration. Streptavidin-coated magnetic particles and biotinylated—Mab 107 antibody were mixed together at the desired concentrations and incubated at 37° C. for ≧1 hr in PBSL buffer. The sample was then washed three times in PBSL buffer to remove any unbound antibody. At the end of the last wash step, the particles were resuspended in 1 ml of the Protein A test sample in PBSL buffer, and incubated at 37° C. for 30 minutes.

### Example 4

### Detection of Non-Particle Bound Protein A in a SAW Sensor

This example demonstrates the detection of Protein A without using magnetic particles. Sensors were prepared as described in Example 1. Dual-channel, 103 MHz, split finger (bidirectional IDT) shear-horizontal surface acoustic wave (SH-SAW) sensors (Sandia National Laboratory) were prepared with RaSa antibody on the active channel and the Chicken IgY on the reference channel. A dual-channel, 113 MHz, SpudT (unidirectional IDT) shear-horizontal surface acoustic wave (SH-SAW) sensor coated with waveguide and supplied by Com Dev was prepared with Mab107 antibody on the active channel and the Chicken IgY on the reference channel.

Separate experiments were run with the Protein A concentrations ranging from 125 ng/ml to 1000 ng/ml. The test sample was injected at time point 150 over the SAW sensor, and the shift in the phase and attenuation difference signals was measured at point 300. The data are shown in Table 1.

TABLE 1
Phase and attenuation responses for Protein A samples
without the use of magnetic particles.

| | Antibody | | | |
|---|---|---|---|---|
| | active | Conc | Phase | Attenuation |
| Sensor | channel | (ng/ml) | response | response |
| Sandia | RaSa | 1000 | 0.50 | 0.03 |
| Sandia | RaSa | 500 | 0.42 | 0.03 |
| Sandia | RaSa | 250 | 0.33 | 0.02 |
| Sandia | RaSa | 125 | 0.13 | 0.01 |
| Com | Mab107 | 1000 | 0.55 | 0.03 |
| Dev | | | | |

The data show relatively small phase and attenuation shifts are produced over the range of Protein A concentrations used in these tests. The lowest concentration of Protein A tested in these experiments, 125 ng/ml, was detectable by relatively small changes (above noise levels) in the phase and attenuation responses of the SAW sensor.

### Example 5

### Detection of Protein A-Biotin Bound to 1 μm Magnetic Particles in a SAW Sensor with a Fixed Magnet in Close Proximity to Sensor Surface

This example demonstrates detection of Protein A-Biotin using a magnetophoresis-based assay when the magnet was raised to the proximity of the sensor surface and maintained in that position for the duration of the experiment. The magnetic particles used in this example were Dynabeads® MyOne™ Streptavidin 1 μm beads (Invitrogen). Sensors were prepared as described in Example 1. Dual-channel, 113 MHz, SpudT (unidirectional IDT) shear-horizontal surface acoustic wave (SH-SAW) sensors coated with waveguide and supplied by Com Dev were prepared with Mab 107 antibody on the active channel and the Chicken IgY on the reference channel.

Streptavidin-coated beads at 1 mg/mL were conjugated with varying amounts of Protein A-biotin and then washed as described in Example 2. The washed beads were further diluted 1:10 (v:v) in PBS and a 500 μl aliquot was used as the test sample for the SAW sensor. A magnet (dimensions and configuration shown in Schematic 1) was fixed directly underneath (i.e., <0.5 mm below) the sensor surface throughout the experiment.

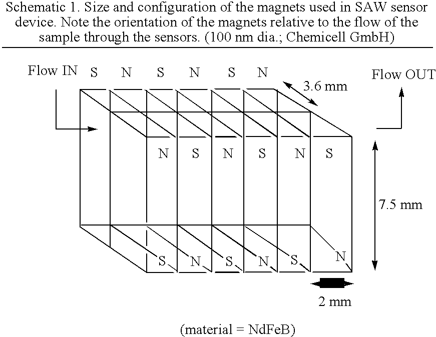

After deposition of the magnetic particles in the sample onto the sensor surface (“sample capture”) was complete, the shift in the attenuation response was measured. The results are shown in Table 2. Using this detection method, the attenuation shift for the binding of 5000 ng/mL Protein A-biotin complex was negligible. This indicates the combination of 1 μm size magnetic particles along with the magnet in close proximity to the sensor does not allow for sensitive (≦5000 ng/mL) detection of Protein A in the sample. These data also show that this detection method (using 1 μm particles and keeping the magnet in close proximity to the sensor) was less sensitive than the method without magnetic particles (Example 4).

TABLE 2
Sensor Response to 1 μm magnetic particles attached to
Protein A - biotin using a process without magnet removal.
The attenuation shift shows the range of results from
four replicate experiments.

| Magnetic particle | Protein A-biotin | | |
|---|---|---|---|
| concentration | concentration | Flow Rate | Attenuation Shift |
| (mg/mL) | (ng/mL) | (ml/min) | (dB) |
| 0.1 | 5000 | 0.03 | 0 to 0.06* |

*Represents the range of results for four replicate experiments

### Example 6

### Effect of Magnet Position on the Detection of Protein A Bound to Sub-Micron Magnetic Particles in a SAW Sensor

Sensors were prepared as described in Example 1. Dual-channel, 113 MHz, SpudT (unidirectional IDT) shear-horizontal surface acoustic wave (SH-SAW) sensors coated with waveguide and supplied by Com Dev were prepared with Mab 107 antibody on the active channel and the Chicken IgY on the reference channel. Magnetic particles coated with streptavidin (100 nm dia.; Chemicell GmbH) were used to measure Protein A biotin in the experimental samples. The 100 nm particles at 1 mg/mL were conjugated with varying amounts of Protein A-biotin and then washed as in Example 2. These particles were then diluted 10-fold in PBSL and a 500 μl volume was used as the test sample for the SAW sensor. For these experiments, the magnet (configured as described in Schematic 1) was fixed directly underneath the sensor (<0.5 mm) and the test sample was injected at time point 150.

No significant response was observed in the phase and attenuation difference signals when the magnet remained in place. The magnet was moved >65 mm away from the sensor at time point 400. In the samples that contained at least 200 ng/mL Protein A, as soon as the magnet was removed, a large (>0.8 dB) response in attenuation was observed (Table 3).

A control experiment was run using the same concentration of magnetic particles, with no Protein A—biotin attached to the particles. The response in attentuation for this experiment was less than 0.2 dB. There was no significant response in phase either after injection or after the magnet was moved away from the sensor. Hence, the large attenuation shifts observed in the 200 ng/ml and the 5000 ng/ml samples indicate that the 100 nm magnetic particles could be used to amplify the signal attenuation, provided the magnet is dropped subsequent to capturing the particle-target complex.

TABLE 3
Sensor response to 100 nm magnetic particles attached to
Protein A - biotin with magnet removal post-capture

| Biotinylated | Magnet distance | | |
|---|---|---|---|
| Protein A conc. | from sensor | Flow Rate | Attenuation shift |
| (ng/ml) | (mm) | (ml/min) | (dB) |
| 0 | 0 | 0.03 | 0.14 |
| 200 | 0 | 0.03 | 0.84 |
| 200 | 0 | 0.03 | 1.21 |
| 5000 | 0 | 0.03 | 1.55 |

The data demonstrate that removing the magnet from the sensor produces an increased sensor response.

### Example 7

### Response of the SAW Sensor to Various Concentrations of Protein A Bound to Sub-Micron Magnetic Particles

This example demonstrates the detection of Protein A using a magnetophoresis-based assay. Dual-channel, 113 MHz, SpudT (unidirectional IDT) shear-horizontal surface acoustic wave (SH-SAW) sensors coated with waveguide and supplied by Com Dev were prepared with Mab107 antibody on both the active and reference channels.

Magnetic particles coated with streptavidin (250 nm dia.; Chemicell GmbH) were used to measure Protein A in the experimental samples. The 250 nm particles at 0.1 mg/mL were conjugated with Protein A samples at different concentrations (0, 2, 20 and 200 ng/ml) as in Example 3. Varying amounts of biotinylated-Mab107 antibody (shown in the table below) were used in each experiment. A 500 μl volume was used as the test sample for the SAW sensor. For these experiments, the magnet (with magnet dimensions and configuration as shown in Schematic 2) was positioned 3 mm underneath the sensor and the test sample was injected over the SAW sensor at time point 50 or 150. The flow path is oriented on the top surface from left to right as shown in the diagram in Schematic 2. Sensor data were collected at the intervals shown in Table 4. At point 200 or 300 the magnet was moved away from the sensor and the shift in the attenuation response measured.

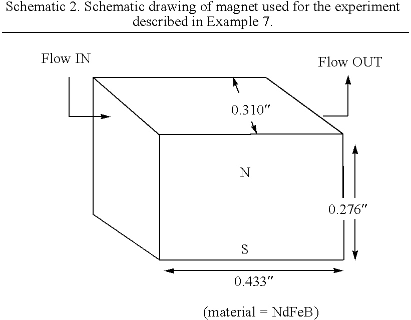

As shown in Table 4, the attenuation response after the magnet was removed was proportional to the concentration of Protein A in the test sample. A concentration of 2 ng/ml was detected in this experiment. This represents significant improvement in the detection limit of Protein A, as compared to an assay using no magnetic particles (Example 4). In these experiments, the largest attenuation response observed for the 20 ng/mL Protein A samples was achieved using the lowest antibody concentration (0.01 μg/ml).

TABLE 4
Sensor response to 250 nm magnetic particles attached to Protein A with magnet
removal post-capture

| | | | Sample | Drop | Magnet | | |
|---|---|---|---|---|---|---|---|
| | Data | | Injection | magnet | distance | | |
| Protein A | Interval | Ab conc | time | Time | from sensor | Flow Rate | Attenuation |
| (ng/ml) | (sec) | (μg/ml) | point | point | (mm) | (ml/min) | shift (dB) |
| 2 | 8 | 0.01 | 150 | 300 | 3 | 0.08 | 0.29 |
| 20 | 8 | 0.01 | 150 | 300 | 3 | 0.08 | 0.96 |
| 20 | 8 | 0.1 | 50 | 200 | 3 | 0.08 | 0.5 |
| 0 | 13 | 1 | 150 | 300 | 3 | 0.08 | −0.06 |
| 20 | 13 | 1 | 150 | 300 | 3 | 0.08 | 0.14 |
| 200 | 13 | 1 | 150 | 300 | 3 | 0.08 | 2.02 |

All references and publications identified herein are expressly incorporated herein by reference in their entirety into this disclosure. Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method of detecting a target biological analyte, the method comprising:
contacting sample material with magnetic particles, wherein a target biological analyte within the sample material interacts with the magnetic particles such that the target biological analyte is bound to the magnetic particle within the sample material;
providing a system comprising an acousto-mechanical device comprising a detection surface with a capture agent located on the detection surface, wherein the capture agent is capable of selectively attaching the target biological analyte to the detection surface;
contacting the detection surface of the acousto-mechanical device with the sample material;
providing a magnetic field generator capable of providing a magnetic field proximate the detection surface that draws the target analyte with the attached magnetic particles to the sensor surface;
selectively attaching the target biological analyte with the attached magnetic particles to the detection surface;
disabling the magnetic field generator to substantially reduce the magnetic field proximate the detection surface; and
operating the acousto-mechanical device to detect the attached target biological analyte while the detection surface is submersed in liquid.

2. A method according to claim 1, wherein the acousto-mechanical device comprises a surface acoustic wave device.

3. A method according to claim 2, wherein the surface acoustic wave device comprises a shear horizontal surface acoustic wave device.

4. A method according to claim 1, wherein the magnetic particles have an average particle size of less than one micron.

5. A method according to claim 1, further comprising contacting the target analyte with a fractionating agent.

6. A method according to claim 1, wherein the disabling of the magnetic field generator comprises removing the magnetic field generator a sufficient distance to substantially reduce the magnetic field proximate the detection surface.

7. A method of detecting a target biological analyte, the method comprising:
fractionating target biological analyte located within sample material;
contacting the fractionated target biological analyte with magnetic particles, wherein the fractionated target biological, analyte within the sample material interacts with the magnetic particles such that the fractionated target biological analyte is bound to the magnetic particle within the sample material;
providing a system comprising a surface acoustic wave sensor comprising a detection surface with a capture agent located on the detection surface, wherein the capture agent is capable of selectively attaching the target biological analyte to the detection surface;
contacting the detection surface of the surface acoustic wave device with the sample material;
providing a magnetic field generator capable of providing a magnetic field proximate the detection surface that draws the target analyte attached to the magnetic particles to the sensor surface;
selectively attaching the target biological analyte to the detection surface;
removing the magnetic field generator a sufficient distance from the detection surface to substantially reduce the magnetic field proximate the detection surface; and
operating the surface acoustic wave sensor to detect the attached fractionated target biological analyte while the detection surface is submersed in liquid.

8. A method according to claim 7, wherein the fractionating comprises chemically fractionating the target biological analyte in the sample material.

9. A method according to claim 7, wherein the fractionating comprises mechanically fractionating the target biological analyte in the sample material.

10. A method according to claim 7, wherein the fractionating comprises thermally fractionating the target biological analyte in the sample material.

11. A method according to claim 7, wherein the fractionating comprises electrically fractionating the target biological analyte in the sample material.

12. A method according to claim 7, wherein the surface acoustic wave sensor comprises a Love mode shear horizontal surface acoustic wave sensor.

13. The method according to claim 1, wherein the target biological analyte comprises bacterial cells.

14. The method of claim 13, wherein the bacterial cells comprise Staphylococcus aureus.

15. The method according to claim 1, wherein the capture agent is an antibody.

16. The method of claim 1, wherein the capture agent is the monoclonal antibody Mab-107.

17. The method of claim 1, wherein the target biological analyte can be detected at concentrations of 1 ng per 500 microliters or greater in the sample material.

18. The method of claim 1 wherein the target biological analyte is whole bacterial cells.

19. The method according to claim 7, wherein the target biological analyte comprises bacterial cells.

20. The method according to claim 7, wherein the capture agent is an antibody.

21. The method of claim 7, wherein the capture agent is the monoclonal antibody Mab-107.

22. The method of claim 7, wherein the target biological analyte can be detected at concentrations of 1 ng per 500 microliters or greater in the sample material.
